(19) United States
(12) Patent Application Publication
(10) Pub. No.: US 2024/0186017 A1
(43) Pub. Date: Jun. 6, 2024

(54) Systems, Methods and Apparatus for Predicting Hemodynamic Events

(71) Applicant: Ottawa Heart Institute Research Corporation, Ottawa (CA)

(72) Inventor: Louise Ying Pw SUN, Gloucester (CA)

(73) Assignee: Ottawa Heart Institute Research Corporation, Ottawa (CA)

(21) Appl. No.: 18/438,599

(22) Filed: Feb. 12, 2024

Related U.S. Application Data
(63) Continuation of application No. PCT/CA2022/051085, filed on Jul. 12, 2022.
(60) Provisional application No. 63/232,337, filed on Aug. 12, 2021.

(51) Int. Cl. G16H 50/30; G16H 10/60

(52) U.S. Cl. CPC G16H 50/30; G16H 10/60

(57) ABSTRACT

A system and method are provided for predicting hemodynamic events. The method includes receiving current patient data, obtaining a current mean arterial pressure (MAP) value from the current patient data, and obtaining prior MAP data obtained from the patient during a period of care. The method also includes using the current MAP value, the past MAP data, a predetermined MAP threshold, and at least one trained model to predict a hemodynamic event, each trained model corresponding to a prediction interval to determine whether the hemodynamic event is expected to occur within a window of time; and outputting an alert indicative of the hemodynamic event to a device configured to display a graphical user interface comprising the alert.

### CROSS-REFERENCE TO RELATED APPLICATIONS

This application is a Continuation of PCT Application No. PCT/CA2022/051085 filed on Jul. 12, 2022, which claims priority to U.S. Provisional Patent Application No. 63/232,337 filed on Aug. 12, 2021, the entire contents of which are incorporated herein by reference in their entirety.

### TECHNICAL FIELD

The following generally relates to systems, methods and apparatus for predicting hemodynamic events such as hypotension, critical hypertension, or cardiac arrest, for example by monitoring patient data and providing a user interface and alerts related to such monitoring and predictions.

### BACKGROUND

The association between hypotension and end organ injury has long been established. However, there has been a lack of consensus on the minimum acceptable mean arterial pressure (MAP) in patients who undergo surgeries requiring cardiopulmonary bypass (CPB).

Hypotension has been found to occur in up to 99% of patients who present for cardiac and major non-cardiac surgery or require intensive care unit (ICU) care. It is associated with mortality and major morbidity such as stroke, acute kidney injury, renal replacement therapy, and myocardial injury.

A recent randomized controlled trial reported that preventing intraoperative hypotension could reduce the risk of postoperative end organ dysfunction by 25%, suggesting that the association between hypotension and organ injury is at least partially causal, and therefore amenable to intervention (see Ref [1]). Hypotension is found to be one of few modifiable perioperative risk factors that if predicted early enough, may be prevented through etiology-specific interventions (see Ref [2]).

The current treatment strategy for hypotension is reactive rather than preventative, however accurate prediction of critical hypotensive events could mitigate complications and potentially improve patient outcomes through proactive management (see Ref [2]).

### SUMMARY

Described herein is a prediction algorithm that can be configured to generate and output personalized minute-by-minute MAP predictions with a high degree of accuracy. The predictions can be displayed in a graphical user interface of an existing or custom device or apparatus to provide alerts to warn of impending, clinically significant hemodynamic events such as impending hypotension or hypertension events or cardiac arrest. Such alerts can be provided at, for example, 5, 10, 15, 20 and 30 minutes prior to onset.

In one aspect, there is provided a system for predicting hemodynamic events, the system comprising: a processor; a communications module coupled to the processor; and a memory, the memory storing one or more trained models and computer executable instructions for predicting hemodynamic events and generating alerts to be displayed in a user interface, the computer executable instructions comprising instructions that when executed by the processor cause the system to: receive via the communications module, current patient data; obtain a current mean arterial pressure (MAP) value from the current patient data; obtain past prior MAP data obtained from the patient during a period of care; use the current MAP value, the past MAP data, a predetermined MAP threshold, and at least one trained model to predict a hemodynamic event, each trained model corresponding to a prediction interval to determine whether the hemodynamic event is expected to occur within a window of time; and output an alert indicative of the hemodynamic event to a device configured to display a graphical user interface comprising the alert.

In another aspect, there is provided a method of predicting hemodynamic events, the method comprising: receiving current patient data; obtaining a current mean arterial pressure (MAP) value from the current patient data; obtaining past prior MAP data obtained from the patient during a period of care; using the current MAP value, the past MAP data, a predetermined MAP threshold, and at least one trained model to predict a hemodynamic event, each trained model corresponding to a prediction interval to determine whether the hemodynamic event is expected to occur within a window of time; and outputting an alert indicative of the hemodynamic event to a device configured to display a graphical user interface comprising the alert.

In another aspect, there is provided a non-transitory computer readable storage medium storing computer executable instructions for predicting hemodynamic events, the computer executable instructions comprising instructions for: receiving current patient data; obtaining a current mean arterial pressure (MAP) value from the current patient data; obtaining past prior MAP data obtained from the patient during a period of care; using the current MAP value, the past MAP data, a predetermined MAP threshold, and at least one trained model to predict a hemodynamic event, each trained model corresponding to a prediction interval to determine whether the hemodynamic event is expected to occur within a window of time; and outputting an alert indicative of the hemodynamic event to a device configured to display a graphical user interface comprising the alert.

### BRIEF DESCRIPTION OF THE DRAWINGS

Embodiments will now be described with reference to the appended drawings wherein:

FIG. 1 is a schematic diagram of a networked computing environment in which a hemodynamic monitoring and alert system operates remotely from patient care/hospital sites and clinician/caregiver devices to obtain patent data, generate a monitoring user interface, and send alerts.

FIG. 2 is a schematic diagram of the hemodynamic monitoring and alert system deployed in a local network at a patient care/hospital site.

FIG. 3 is a schematic diagram of the hemodynamic monitoring and alert system integrated into a bedside unit.

FIG. 4 is a schematic diagram of the hemodynamic monitoring and alert system integrated into a personal/wearable unit.

FIG. 5 is a perspective view of a monitoring unit with user interface for monitoring data and alerts generated by the hemodynamic monitoring and alert system.

FIG. 6 is a schematic block diagram of an example of a configuration for the hemodynamic monitoring and alert system and a model training system configured to generate trained models to be used by the hemodynamic monitoring and alert system.

FIG. 7 is a schematic block diagram of an example of a configuration for a client device equipped to communicate with the hemodynamic monitoring and alert system.

FIG. 8 is a flow chart illustrating computer executable instructions for generating trained models for MAP prediction.

FIG. 9 is a flow chart illustrating computer executable instructions for analyzing patient data to generate MAP predictions and alert patients of hemodynamic events.

FIG. 10 is a screen shot of a hypotension monitoring graphical user interface showing an alert with a five minute prediction window.

FIG. 11 is a screen shot of a hypotension monitoring graphical user interface showing an alert with a ten minute prediction window.

FIG. 12 is a screen shot of a hypotension monitoring graphical user interface showing an alert with a fifteen minute prediction window.

### DETAILED DESCRIPTION

Critical thresholds of hypotension during physiologically distinct stages of cardiac surgery have been identified (i.e., pre-, during, and post-bypass (or CPB)) in association with postoperative stroke, acute kidney injury, renal replacement therapy, and mortality (see Refs [2]-[5]). Specifically, it was found that MAP <65 mmHg during bypass is associated with an increased risk of stroke (see Ref [3]), whereas MAP <65 mmHg after bypass is associated with an increased risk of renal replacement therapy and death in a dose-dependent fashion (see Refs [4], [5]). To leverage these findings, the following describes a system, method and apparatus, including machine learning algorithms used with same to predict the onset of critical hemodynamic events, such as hypotensive events during cardiac surgery and postoperatively in the ICU.

The system described herein includes a prediction algorithm that can be configured to generate and output personalized minute-by-minute MAP predictions with a high degree of accuracy. The predictions can be displayed in a graphical user interface of an existing or custom device or apparatus to provide alerts (e.g., alarms, push notification functionality, etc.) to warn of impending, clinically significant hemodynamic events. Such alerts can be provided at, for example, 5, 10, 15, 20 and 30 minutes prior to onset.

The present system can be configured specifically for use in an ICU and an operating room (OR)—including both cardiac and non-cardiac ORs, where hemodynamic events such as hypotension are most common. For example, with respect to cardiac surgery, the system has been trained on data from three physiologically distinct phases pre-, during, and post-bypass. The system has also been trained on a large number of cardiac surgical ICU patients and performed very well on external validation in a broader international cohort of medical-surgical ICU patients. The system can therefore be tailored to all high-risk patients, namely the population that could benefit most from using the system.

The system can also predict the actual MAP value and provide a graphical representation of predicted MAP magnitude and duration up to a certain window into the future (e.g., 30 min). This feature advantageously allows the clinician to decide whether the upcoming hemodynamic episode is actionable. For instance, when considering hypotension episodes, it may be safer to maintain the current state during mild episodes (e.g., when MAP falls 1 mmHg below the desired threshold for a short period of time), than to overtreat and cause complications.

The system can also support personalized MAP thresholds. This is also advantageous as MAP goals should be tailored to patient comorbidities, as well as the presenting illness and procedure-specific requirements. The system provides an intuitive and easily interpretable user interface. Settings can be personalized, for example, from presets (e.g., cardiac surgery, noncardiac surgery, ICU) that can be tweaked to tailor to individual needs (e.g., prediction 15-30 min into the future might be more useful for the ICU setting, whereas prediction of future events within 5-10 min may be more relevant in the OR).

As illustrated in greater detail below, the system and methods described herein can be deployed in a wide range of applications, for example, in cardiac and major noncardiac operating rooms or any type of ICU. Specifically, the system and methods can be deployed using a standalone monitoring device, as a software add-on for existing monitoring technology (e.g., OR and ICU monitors, other hemodynamic modules, or as EHRs), and/or as a remote monitoring service (e.g., nursing station monitors, telehealth systems, augmented and/or virtual reality healthcare devices, and other virtual care platforms to monitor individuals or groups of patients simultaneously). When a network connection is available, the system in any of these forms can be configured to push alerts to various client devices used by physicians, clinicians, nurses, other healthcare providers, caregivers, or the patients themselves. The networked functionality of the presently described system is particularly advantageous for monitoring and alerting patients kept in isolation who would normally receive less care/monitoring (e.g., due to recent COVID-19 pandemic restrictions). The remote monitoring and alerts can therefore mitigate potentially catastrophic events in these and other types of patients.

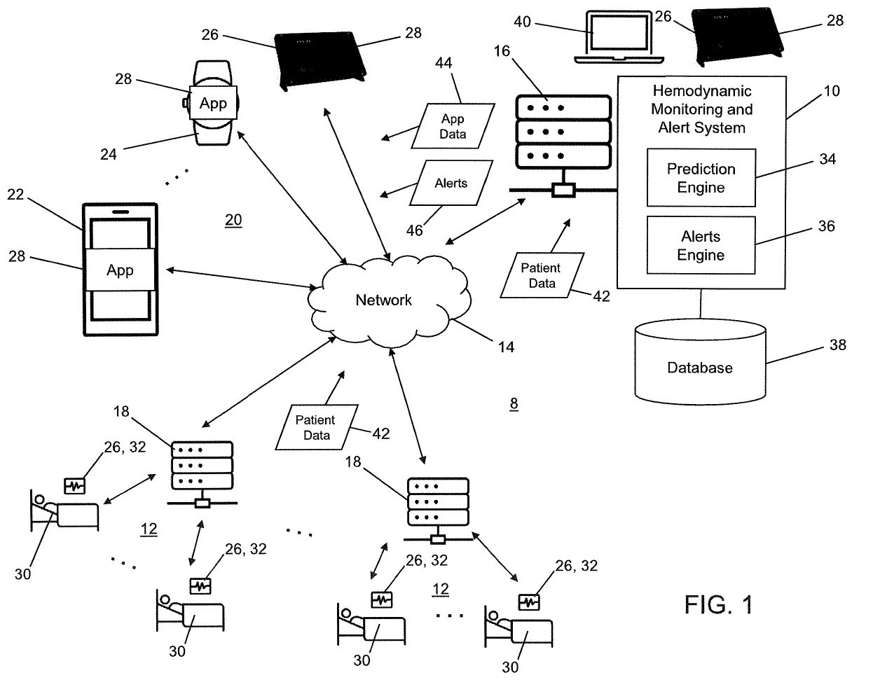

Turning now to the figures, FIG. 1 illustrates a computing environment 8 in which a hemodynamic monitoring and alert system 10 (hereinafter also referred to as “the system 10”) can be deployed to monitor one or more physiological parameters of a subject (e.g., person, patient, user, etc.). In the example shown in FIG. 1, the system 10 is deployed in a networked computing environment 8 in which a number of monitored sites 12 (e.g., patient sites such as ICUs, ORs, critical response units (e.g., vehicles or modules), patient rooms, outpatient clinics, at-home care locations, etc.) are remotely monitored and alerted via a communication network 14 and a server 16 coupled to the communication network 14 to provide network connectivity to the system 10.

Communication network 14 may include a telephone network, cellular, and/or data communication network to connect different types of client devices to the system 10. For example, the communication network 14 may include a private or public switched telephone network (PSTN), mobile network (e.g., code division multiple access (CDMA) network, global system for mobile communications (GSM) network, and/or any 3G, 4G, or 5G wireless carrier network, etc.), WiFi or other similar wireless network, and a private and/or public wide area network (e.g., the Internet). Communication network 14 may also include any short- or long-range communication channel with an ability to send and receive messages, data packets or other data formats/structures, including email, wearable connected devices (e.g., connectable over Bluetooth), pager networks, etc.

In the example shown in FIG. 1, each monitored site 12 can include a local or sub-network with a local server 18 coupled to the network 14 to enable multiple patients 30 to be monitored by the system 10. That is, the local servers 18 are coupled to the communication network 14 to exchange data with the remote server 16 coupled to the system 10.

The system 10 can also use its connectivity via the communication network 14 to communicate with various monitoring sites 20. The monitoring sites 20 can be the same or different from the monitored sites 12. In this way, physicians, clinicians, nurses, caregivers or other entities can be provided with an ability to remotely or otherwise portably monitor a patient at a monitored site 12. In this example, a mobile device 22 (e.g., smartphone, tablet, other computing device), a wearable device 24 (e.g., smartwatch, heads-up display (i.e., smart glasses), monitoring tag, etc.), and a custom console 26 are shown by way of illustration only. It can be appreciated that any suitable “client device” can be equipped with an application (app) 28 to generate and display a graphical user interface associated with data being monitored by the system 10, such as MAP thresholds and predictions related to same. For example, the monitored sites 12 can include bedside monitoring equipment 32 in addition to a customized console 26. That is, existing hardware can be configured to operate a software program that provides the graphical outputs, alerts, etc. as described herein.

The system 10 includes a prediction engine 34 to determine MAP predictions indicative of potential hemodynamic events, such as hypotension events. It can be appreciated that while various examples provided herein may refer to monitoring of hypotension events, the principles and system configuration can be equally adapted to any hemodynamic event such as critical hypertension, cardiac arrest, etc. The system 10 also includes an alerts engine 36 to generate and send alerts to one or more predetermined entities having a suitable client device running an app 28 or other software program, and includes or has access to a database 38. The database 38 can be used by the system 10 to store patient data 42 sent to the system 10 from the patient sites 12. The database 38 can also be used to store models, training data, contact information, and any other data used by the system 10 in generating predictions and alerts. The system 10 can generate app data 44 instructing the app 28 at a remote location what to display, and can generate alerts 46 to be sent to a monitoring site 20 such as a caregiver's smartphone. It can be appreciated that the app data 44 and alerts data 46 are shown separately for illustrative purposes and could instead be sent together in the same data packet, message, or other data structure. As illustrated in FIG. 1, the system 10 can also include a console 26 or other computing terminal 40 to be used by an administrator or caregiver.

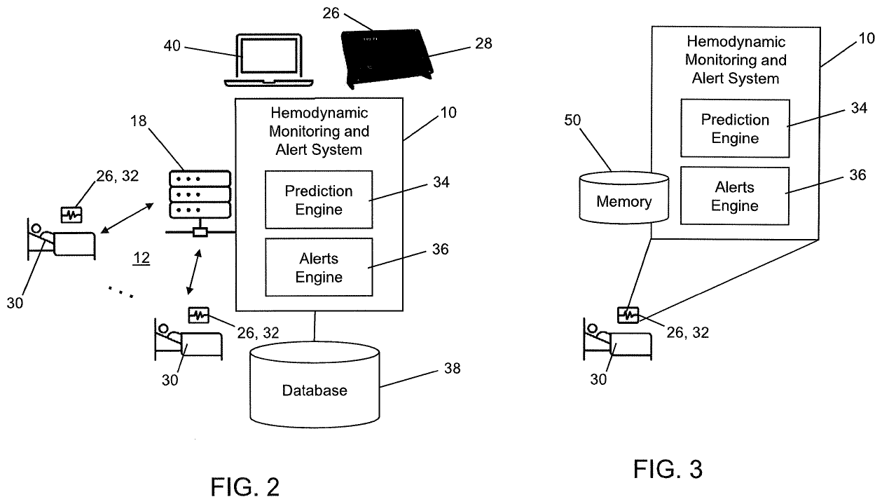

The configuration shown in FIG. 1 provides centralized remote monitoring of multiple monitored sites 12 (e.g., a hub and spoke system for hospitals or healthcare clinics) and the ability to connect with multiple monitoring sites 20. The system 10, however, can be deployed in various other configurations. For example, as shown in FIG. 2, the system 10 can be coupled to a local server 18 at one of the monitored sites 12 such as within an individual hospital or clinic. Here the setup shown in FIG. 2 is replicated in a local setting to monitor multiple patients 30 for a single entity (e.g., a stack of monitoring devices at a nurse's station telemetered to patient rooms). That is, while FIG. 1 illustrates that the same system 10 can be used to monitor patients in multiple sites 12, local deployments at individual sites 12 are also possible. In another configuration example, shown in FIG. 3, the system 10 can be integrated directly into a console 26 or existing bedside equipment 32 for an individualized monitoring system 10. In this example, the system 10 can use a local memory 50 to store data required to perform monitoring and alert functionality.

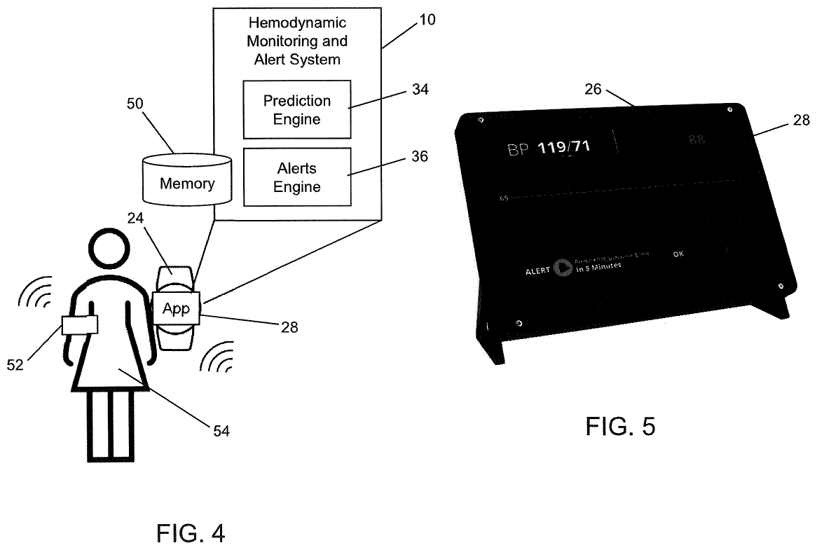

Referring now to FIG. 4, yet another configuration is shown in which the system 10 is embedded or partially deployed in an edge device including personal devices such as a wearable device 24 in this scenario. In this scenario, it is assumed that a user 54 has a wearable monitor 52 that sends data to the system 10 in the wearable device 24 to be displayed using the app 28. In the configuration shown in FIG. 4, the system 10 could be embedded in the wearable device 24 or can be centrally or otherwise remotely deployed as in FIG. 1 with the wearable device 24 used to collect patient data 42 from the wearable monitor 52 and relay such data 42 to the system 10 for analyzing and processing data and to push out alerts 46. It can also be appreciated that a wearable device 24 could be associated with a caregiver or clinician such that the user 54 employs the wearable monitor 52 and a wearable device 24 to collect and send patient data 42 to the system 10 while another entity performs the monitoring and/or receives the alerts 46.

FIG. 5 illustrates an enlarged view of the console 26 that includes a display for a graphical user interface employed by the app 28. It can be appreciated that the console shown in FIG. 5 is purely illustrative and many other form factors and design elements can be considered. Further detail of the graphical user interface provided by the app 28 is described later.

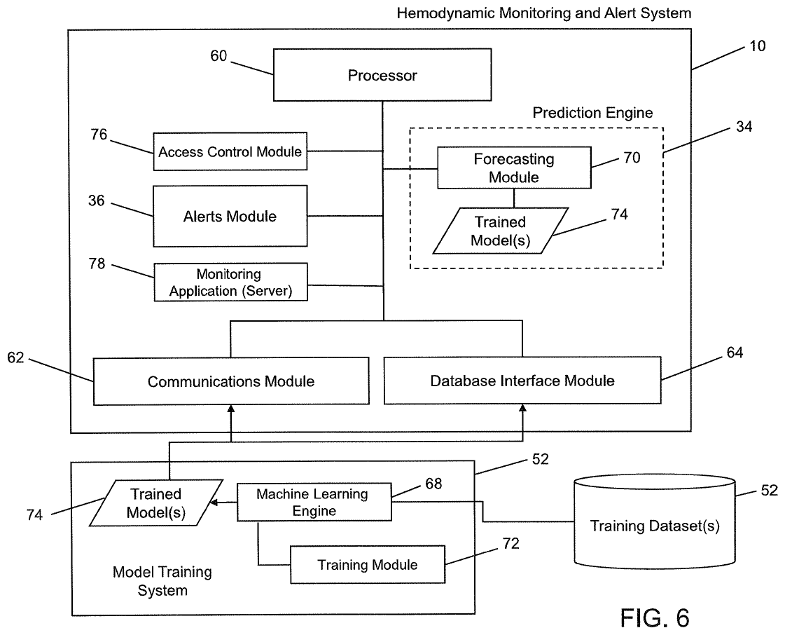

In FIG. 6, an example of a configuration for the system 10 and a separate model training system 52 are shown. In certain embodiments, the system 10 may include one or more processors 60, a communications module 62, and a database interface module 64 for interfacing with the database 38 or memory 50 to retrieve, modify, and store/add data. The database interface module 64 can also be used to send or receive data to/from the model training system 52. Communications module 62 enables the system 10 to communicate with one or more other components of the computing environment 8, such as client devices (or one of its components) or model training system 52, via a bus or other communication network, such as the communication network 14. While not delineated in FIG. 6, the system 10 includes at least one memory or memory device that can include a tangible and non-transitory computer-readable medium having stored therein computer programs, sets of instructions, code, or data to be executed by processor 60. FIG. 6 illustrates examples of modules, tools and engines stored in memory by the system 10 and operated by the processor 60. It can be appreciated that any of the modules, tools, and engines shown in FIG. 6 may also be hosted externally and be available to the system 10, e.g., via the communications module 62.

In the example embodiment shown in FIG. 6, the system 10 includes the prediction engine 34 and alerts module 36 shown in FIGS. 1-4. The prediction engine 34 in this example includes a forecasting module 70 and one or more trained models 74. The prediction engine 34 can leverage the processing performed by the model training system 52 to utilize the forecasting module 70 and one or more trained models 74 to make MAP predictions and generate a UI and alerts for a client device 12. For example, the prediction engine 34 can store predictive algorithms for scenarios/applications such as the OR and/or ICU for each prespecified time period. For example, the system 10 can store two groups of models 74, one for each of the OR and ICU. Each of these groups of models 74 can contain individual models 74 for each of the predictive timeframes (e.g., 5, 10, 15, 20, 30 mins).

The model training system 52 can be a separate entity or otherwise coupled to the system 10 to perform the training prior to deployment and use of the models 74 in the system 10 for ongoing monitoring. The model training system 52 includes a machine learning engine 68, a training module 72, and is configured to generate the trained models 74 to be deployed to the system 10 as described in greater detail below. It can be appreciated that if/when the trained models 74 are refined or replaced with newer models 74 by the training system 52, the training system 52 can communicate with the system(s) 10 to have them update the locally-stored trained models 74. It can be appreciated that the model training system 52 can be implemented as a federated learning system that is configured to continuously train and improve the models 74 in real-time and update the system 10. The system 10 also includes an access control module 76 and a server-side monitoring application 78.

The prediction engine 34 can utilize or otherwise interface with the forecasting module 70 to forecast MAP values based on current and previous MAP that is continually being processed using the trained models 74. The MAP values can be obtained according to the EHR protocols and infrastructure being used. For example, MAP values can be decoded and extracted from HL7 messages (described further below) as a data processing/preparation step. The clean, processed data can then be fed into the model 74. Different data processing steps may be implemented depending on the EHR system and in some cases the MAP data can be extracted directly from patient monitors before the data reaches the EHR. The trained models 74 are generated by the training system 52 using the machine learning engine 68 using one or more machine learning algorithms. The machine learning algorithms may include, but are not limited to, a recurrent neural network model, and the one or more machine learning algorithms may be trained against, and adaptively improved Patient parameters may be stored and maintained using the forecasting module 70.

The trained model(s) 74 may also be created, refined, updated, and re-trained by the training system 52, communicated to the system 10, and stored and referenced by the prediction engine 34 in various functions, services, or applications utilized or provided by the system 10. In some instances, data stored in the forecasting module 70 may identify one or more parameters, that facilitate a prediction of upcoming MAP values based on any of the exemplary machine learning algorithms or processes described herein. The one or more forecasting parameters may correspond to MAP parameters that can indicate an affinity or compatibility between groups of patient data 42 and certain potential actions. For example, certain patient data 42 can be indicative of an impending hypotension event within an alert interval such as the 5, 10, 15, 20 and 30 minute intervals noted above. Examples of adaptive machine learning processes include, but are not limited to, one or more artificial, recurrent neural network models, such as an LSTM model, e.g., implemented using a corresponding neural network library, such as Keras® and PyTorch.

Based on the output of the one or more machine learning algorithms or processes, such as the recurrent neural network model described herein, machine learning engine 68 may perform operations that enable MAP values to be predicted using current and previous MAP data. The outputs of the machine learning algorithms or processes, i.e., the trained model(s) 74 may then be provided to, stored, and used by the prediction engine 34 to generate one or more suggested actions that can be presented to the client device or to present certain data within the app 28.

Referring again to FIG. 6, the access control module 76 may be used to apply a hierarchy of permission levels or otherwise apply predetermined criteria to determine what patient data 42 can be shared with which entity in the computing environment 8. For example, the system 10 may have been granted access to certain sensitive patient data 42 while certain individuals being provided with app data 44 and/or alerts 46 may not. As such, the access control module 76 can be used to control the sharing of certain patient data 42 based on a type of client/user, a permission or preference, or any other restriction imposed by the computing environment 8 or application in which the system 10 is used. Other mechanisms can be employed for addressing data sensitivity, such as deidentifying patients at the source of the incoming data. Moreover, it can be appreciated that data can be obtained directly from the device which collects the patient data 42 or an intermediary server where issues of versatility, reliability and/or security are considered.

The server-side monitoring application 78 can be used by the system 10 to provide a remote monitoring dashboard or master application to be displayed to an administrator or can operate as a server-based application to exchange data with the monitored sites 12 and monitoring sites 20 and to onboard additional sites 12, 20 or patients 30.

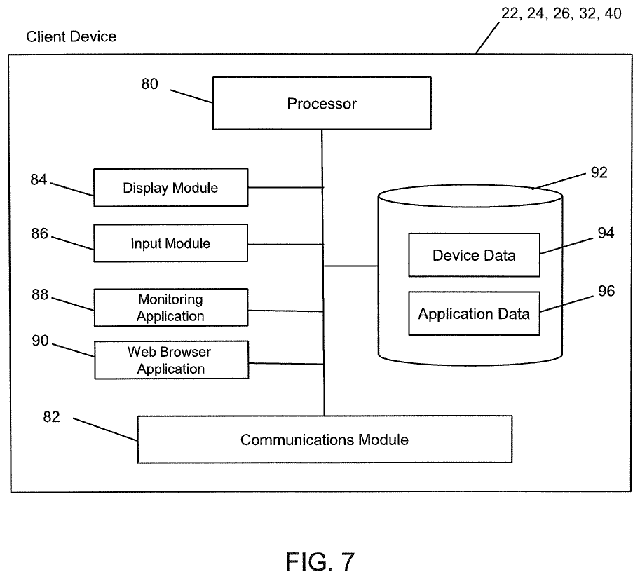

In FIG. 7, an example configuration of a client device is shown, for example any one of the devices 22, 24, 26, 32, 40 shown in FIGS. 1-4. In certain embodiments, the client device may include one or more processors 80, a communications module 82, and a data store 92 storing device data 94 and application data 96. Communications module 82 enables the client device to communicate with one or more other components of the computing environment 8, such as the system 10, via a bus or other communication network, such as the communication network 14. While not delineated in FIG. 7, the client device includes at least one memory or memory device that can include a tangible and non-transitory computer-readable medium having stored therein computer programs, sets of instructions, code, or data to be executed by processor 80. FIG. 7 illustrates examples of modules and applications stored in memory on the client device and operated by the processor 80. It can be appreciated that any of the modules and applications shown in FIG. 7 may also be hosted externally and be available to the client device, e.g., via the communications module 82.

In the example embodiment shown in FIG. 7, the client device includes a display module 84 for rendering GUIs and other visual outputs on a display device such as a display screen, and an input module 86 for processing user or other inputs received at the client device, e.g., via a touchscreen, input button, transceiver, microphone, keyboard, biometrics, gestures etc. It can be appreciated that MAP values can be obtained from various input devices, such as infrared sensors or pulse wave monitors from wearable devices. For example, non-invasive MAP values could be obtained from smartphone apps, smart watches, personal fitness monitors, telemonitoring wearable devices, as well as other non-invasive hemodynamic monitors.

The client device may also include a monitoring application 88 provided by an administrator of the system 10, e.g., for performing patient monitoring and/or to receive alerts 46. The monitoring application 88 can be configured to receive the app data 44 from the system 10. The client device in this example embodiment also includes a web browser application 90 for accessing Internet-based content, e.g., via a mobile or traditional website. The data store 92 may be used to store device data 94, such as, but not limited to, an IP address or a MAC address that uniquely identifies client device within environment 8. The data store 92 may also be used to store application data 96, such as, but not limited to, login credentials, user preferences, cryptographic data (e.g., cryptographic keys), etc. As discussed above, the system 10 can be deployed in several configurations. Depending on the configuration, data storage and processing functionality can be provided at the level of the individual bedside device or at the level of the central processor 60 in the system 10, e.g., in the case of telemonitoring or edge device applications.

It will be appreciated that only certain modules, applications, tools and engines are shown in FIGS. 1 to 7 for ease of illustration and various other components would be provided and utilized by the system 10 and client devices illustrated in FIGS. 1-4, as is known in the art.

The prediction engine 34 in the system 10 enables individualized hypotension prediction algorithms to be deployed as software in existing systems and/or as software in a hardware unit such as the console 26. In one example, a hypotension prediction algorithm utilized by the prediction engine 34 predicts critical hypotensive events up to a certain interval ahead of time (e.g., up to 30 minutes). The software program utilized by the prediction engine 34 in this example, can include two parts, namely i) an intraoperative (OR) algorithm trained and validated on data from patients who underwent major cardiac surgery during a prior period of time; and ii) an ICU-specific algorithm trained on ICU data from cardiac surgery patients during a prior period of time. The two algorithms to use the previously-trained models 74 have been externally validated on both cardiac surgery patients and a mixed medical-surgical ICU cohort from the MIMIC-Ill dataset. The MIMIC Ill dataset is an open-source, anonymized dataset that comprises of hemodynamic data from general ICU admissions at a tertiary U.S. center (see Ref [6]). The algorithm's performance, evaluated using the Root Mean Square Error (RMSE), Mean Average Error (MAE), and ratio of predicted hypotension episodes over actual hypotension episodes (R_{P/A}) metrics, is highlighted in Table 1 below.

TABLE 1
Performance of the OR and ICU Hypotension Prediction Models

| | | Prediction | RMSE | MAE | RP/A | RMSE | MAE | RP/A |
|---|---|---|---|---|---|---|---|---|
| Setting | Phase | Timeframe | (UOHI) | (UOHI) | (UOHI) | (MIMIC) | (MIMIC) | (MIMIC) |
| OR | Pre-Bypass | 5 min | 11.29 | 4.49 | 90.13% | N/A | N/A | N/A |
| | Bypass | 5 min | 7.23 | 3.09 | 91.37% | N/A | N/A | N/A |
| | Post-Bypass | 5 min | 7.55 | 2.9 | 90.79% | N/A | N/A | N/A |
| ICU | 12 h post-op | 1 min | 9.39 | 3.53 | 97.26% | 10.45 | 3.16 | 97.67% |
| | 12 h post-op | 5 min | 10.68 | 4.77 | 92.59% | 13.07 | 5.65 | 94.57% |
| | 12 h post-op | 10 min | 11.56 | 5.6 | 67.97% | 14.39 | 6.48 | 92.40% |
| | 12 h post-op | 15 min | 11.85 | 5.97 | 75.94% | 15.01 | 6.43 | 90.65% |

Potential applications include direct EHR integration, integration with established ICU hemodynamic monitors, and other monitoring systems and client devices (e.g., operating room monitors, cardiac output monitors, critical care or ambulance transport monitors, wearables, augmented reality devices, etc.). The system 10 could also be used as a standalone module, or as a remote monitoring software with the capability of pushing critical warnings to healthcare providers as illustrated in FIGS. 1 and 2 in both globally and locally deployed networked environments. As discussed above, the system 10 can be deployed in a custom console 26 that can be mounted at the bedside.

### Model Training Phase:

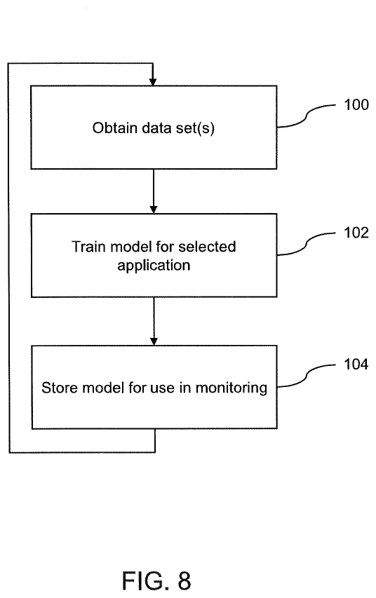

Referring now to FIG. 8, a process used by the training system 52, for generating the trained models 74 is shown. At step 100 one or more data sets is obtained. The data set(s) is/are used at step 102 to train the model for a selected application, e.g., OR, ICU, etc. The model is then stored at step 104 and provided to the system 10, to enable the prediction engine 34 to utilize the forecasting module 70 in a real-time monitoring scenario.

To generate an OR model as one of the trained models 74 used by the prediction engine 34, data was extracted from an electronic patient record system (e.g., CompuRecord, Philips Medical Systems, The Netherlands), which included intraoperative hemodynamic and medication administration data on adult patients who underwent major cardiac surgery requiring cardiopulmonary bypass at the University of Ottawa Heart Institute (UOHI) between November 2009 and March 2015. All intraoperative invasive blood pressure measurements were recorded automatically every 15 seconds and medication administration at the point of care. Patients who underwent heart transplant, ventricular assist device implant, and off pump procedures were excluded, as were patients who did not have complete MAP data at a minimum of 1 min intervals. This was done to avoid inclusion of artificial values through imputation. A set of unique patient records that were available were used for training and internal validation of the OR model. It can be appreciated that different sets of patient records could be used to retrain the model(s) 74 at a later time. Such retrained models 74 could then be deployed as “updates” to the system(s) 10 in the field being used for ongoing monitoring.

In this example training operation, the intraoperative observation window was capped at 11.5 hours, commencing at the time of arterial line insertion (i.e. anesthesia induction), and spanned the pre-bypass, bypass, and post-bypass phases of surgery.

To generate an ICU model as one of the trained models 74, data was extracted from the EPIC EHR system, at a sampling rate of 1 per minute and in the form of HL7 messages. HL7 is a data exchange format that is used for exchanging, retrieving, and integration of electronic medical and administrative information in the healthcare sector. HL7 stands for Health Level 7, which is a set of standards for communication between healthcare providers. In this example, all postoperative cardiac surgical intensive care unit (CSICU) admissions between Jul. 17, 2019 and January 2021 at the UOHI, with a minimum length of stay of 8 hours, were included. CSICU readmissions were excluded, if occurring during the same hospitalization episode. A set of unique patient records that were available were used for the training and internal validation of the ICU model.

The ICU model was externally validated using a set of unique general (i.e., mixed medical and surgical, including cardiac surgery) ICU patient records from the MIMIC Ill dataset, which was sampled at 1 min intervals to match the UOHI ICU sampling frame rate. To ensure generalizability of the ICU model, the MIMIC Ill validation dataset was not restricted to patients receiving cardiac surgery, but rather included the entire cohort of pediatric to adult patients who were admitted to the ICU for a minimum duration of 8 hours.

In this example, the ICU observation window included 11.5 hours of hemodynamic data during the first 12 hours of a patient's ICU stay. Only numeric MAP data was used in the model derivation.

Separate models 74 for the OR and the ICU were generated, in order to tailor to the differences in physiology and hemodynamic signatures of these specialized environments. Specifically, intraoperative hemodynamics are “rockier” than the ICU, with more sudden episodes of hypotension, some of which are unpredictable (i.e., sudden surgical blood loss; lifting of the heart to check anastomosis and hemostasis; sudden onset ischemia and dysrhythmia). The cardiopulmonary bypass period is also unique to the cardiac OR. During this period of non-pulsatile flow, full body perfusion is provided by the heart and lung machine. Both the bypass and post-bypass period are critical for end organ injury. In the latter, the heart is restarted after a period of ischemia and arrest, and sometimes struggles to function adequately to balance the body's metabolic supply and demand.

For these reasons, the hypotension model 74 was retrained for each of the following phases:
OR: pre-bypass, bypass, and post-bypass, and
ICU: the first 11.5 hours after surgery.

MAP data from CompuRecord was recorded at a sampling frequency of 15 seconds. MAP and intraoperative drug data were re-sampled at a 1-minute frequency (taking the median value for that minute) and queried from the vitals table. The events table was queried for bypass time, whereby the timestamps were used to segment the data into pre-bypass, bypass, and post-bypass phases. In cases where these timestamps were missing, a pulsatility detection algorithm was used to estimate bypass initiation and end times with good accuracy.

In this example a set of 6,410 unique patients were considered with the first 695 MAP values for each record. With a 1 min sampling frequency, the total OR duration studied amounted to 11.5 hours per patient record.

For ICU data preparation, MAP data was sampled at a 1-minute frequency and queried from EPIC HL7 messages. 2,122 unique ICU patients were considered with the first 695 MAP values in each record. Since ICU data is sampled at 1 min frequencies, a total of 11.5 hours of MAP data were considered per patient record.

When training the models 74, the same artifact removal process was considered for both the OR and ICU models.

### Artifact Removal:

Each MAP value was compared to the next eight values, and if the change was greater than 50%, it would be replaced with the previous value. This mechanism was developed to smooth out any technical artifacts that would occur due to arterial line flushing or transducer dislocation.

For extreme, non-persistent MAP values (spikes) that were not indicative of tachycardia or cardiac arrest: If the highest value was above 150 mmHg and not preceded or followed by a value within 30% of it, it was replaced with a maximum MAP of 150 mmHg. If the lowest value was below 30 mmHg and not preceded or followed by a value within 30% of it, it was replaced with a minimum MAP of 30 mmHg.

### Pressure LSTM Model:

To train the models 74 to enable the prediction engine 34 to predict the MAP, a deep learning long-short term memory (LSTM) model was constructed and used by the training system 52, using longitudinal sequential patient level data that has been collected over time across the entire monitored period. The deep learning sequential model, LSTM developed in this research study requires a sequence of hemodynamic data points in the observation window, unlike traditional machine learning models. Traditional classification models require one record per patient containing the predictors (independent variables) and a response variable (dependent variable) which is a binary variable.

The MAP was predicted using a Pressure LSTM model, which was developed using the Python PyTorch library, with only numeric MAP values to predict the next-minute MAP (one dimensional vector). An 80-20 train-test split was adopted.

### Experimental Runs:

Each experimental scenario was run 10 times, to minimize the effect of the random varying initial conditions of the LSTM network which could result in very different results each time a given configuration is trained.

A diagnostic approach was used to investigate model configurations, where plots of training epochs were created and studied for insight into how a given configuration performs and how it may be adjusted to elicit better performance.

The model was evaluated on both the train and test datasets at the end of each epoch, and the train and test RMSE scores were saved at the end of each scenario to indicate progress.

The following parameters were configured, and the model was run 10 times for each combination of parameters:
different weights.
different learning rates.
different number of hidden layers.
bidirectional vs unidirectional LSTM
different number of inputs.
different prediction windows: 1 minute, 5 minutes, 10 minutes, 15 minutes, and 30 minutes
intraoperative drugs as additional input (only for the OR model)

The LSTM network was initially fit for 100 epochs. The lowest RMSE value was obtained with 50 epochs and a batch size of 100, which was chosen for the final Pressure LSTM model.

The final model was trained separately on the OR and ICU datasets, considers only the MAP as variable, and is fit using the efficient Adam version of stochastic gradient descent and optimized using the mean squared error, or ‘mse’ loss function.

The Root Mean Square Error (RMSE), Mean Average Error (MAE), and ratio of predicted to observed hypotension episodes were the metrics used to compare model performance for each prediction window.

### Model Deployment and Monitoring:

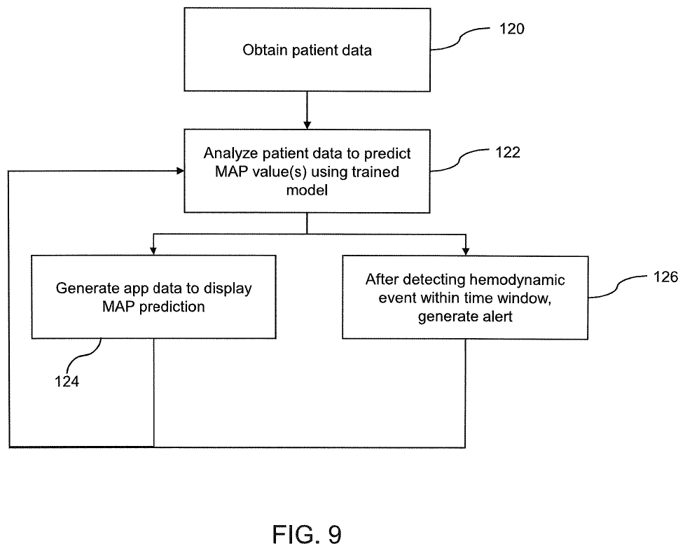

The trained model(s) 74 can be stored by the system 10 and used by the forecasting module 70 of the prediction engine 34 for ongoing monitoring to predict MAP values and hemodynamic predictions at the predetermined intervals such as 5, 10, 15, 20, 30 mins prior to a hemodynamic event. Referring now to FIG. 9, a flow chart is provided to illustrate operation of the system 10 to use the trained model(s) to monitor patient data 42, determine a MAP prediction, and display app data 44 and, when appropriate, generate an alert. At step 120, patient data 42 is obtained from a patient, e.g., from a monitored site 12. The patient data 42 is processed and analyzed at step 120 to extract the current MAP value and, using the trained model 74, use past MAP data to predict the MAP at a later time. For example, a window_size=1 can be used such that at every step, the input at t−1 is taken into consideration, along with any hidden states (i.e., any memory of past observations), for the algorithm to look at the past minute of actual data to forecast the next minute. At steps 124 and 126, the app data 44 and alert 46 are generated to enable the app 28 to display a MAP prediction. In step 122, the forecasting module 70 runs the models for each interval simultaneously. If a hypotension event is forecast at 5 minutes out, a corresponding alarm is output. If hypotension is forecast to occur at both 10 and 15 minutes, another alarm can be output, etc. The alarms can be configured to provide an alert for the most imminent episode of deterioration, e.g., 10 minutes in this example.

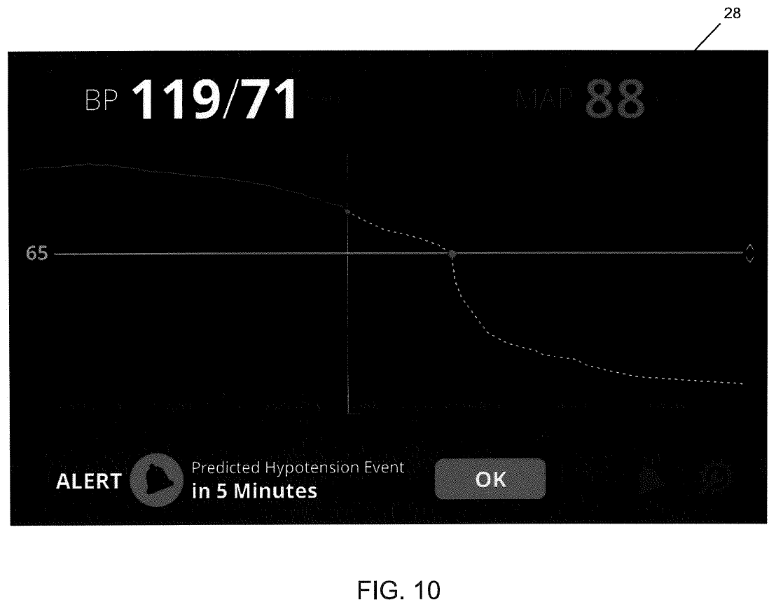

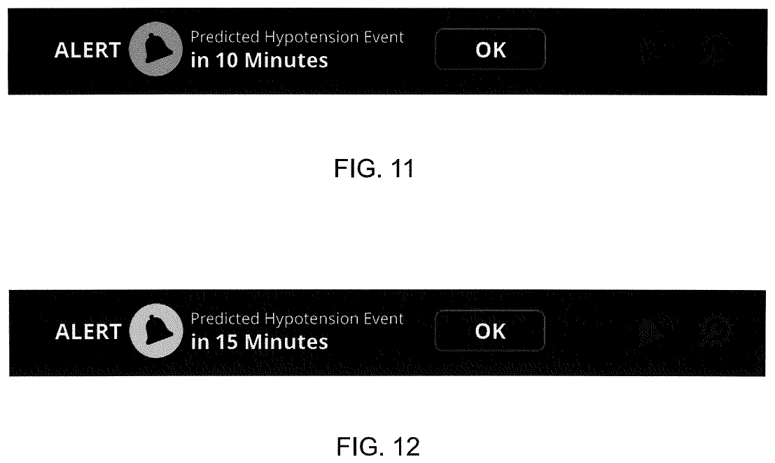

FIGS. 10-12 illustrate screen shots of graphical user interfaces displayed by the app 28. In the user interfaces, the blood pressure (BP) is displayed along with the current MAP value. A time scale is shown with a personalized map target line and a vertical “now” line crosses the timeline. The past MAP values are displayed as an evolving graph that crosses the “now” line to show the current MAP value, and is extrapolated out in the dashed line. In FIG. 10 it can be seen that the personalized threshold is expected to be crossed in about 5 minutes. A color-coded alert system can also be displayed. FIG. 10 illustrates a 5 minute window. FIG. 11 illustrates the alert at the 10 minute window, and FIG. 12 illustrates the alert at the 15 minute window. An OK button can be provided to dismiss or silence the alarm. Other user interface options such as volume and settings menus can also be accessible through a touch screen interface.

For simplicity and clarity of illustration, where considered appropriate, reference numerals may be repeated among the figures to indicate corresponding or analogous elements. In addition, numerous specific details are set forth in order to provide a thorough understanding of the examples described herein. However, it will be understood by those of ordinary skill in the art that the examples described herein may be practiced without these specific details. In other instances, well-known methods, procedures and components have not been described in detail so as not to obscure the examples described herein. Also, the description is not to be considered as limiting the scope of the examples described herein.

It will be appreciated that the examples and corresponding diagrams used herein are for illustrative purposes only. Different configurations and terminology can be used without departing from the principles expressed herein. For instance, components and modules can be added, deleted, modified, or arranged with differing connections without departing from these principles.

It will also be appreciated that any module or component exemplified herein that executes instructions may include or otherwise have access to computer readable media such as storage media, computer storage media, cloud storage and processing, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the system 10, devices 22, 24, 26, 32, 40, any component of or related thereto, etc., or accessible or connectable thereto. Any application or module herein described may be implemented using computer readable/executable instructions that may be stored or otherwise held by such computer readable media.

The steps or operations in the flow charts and diagrams described herein are just for example. There may be many variations to these steps or operations without departing from the principles discussed above. For instance, the steps may be performed in a differing order, or steps may be added, deleted, or modified.

Although the above principles have been described with reference to certain specific examples, various modifications thereof will be apparent to those skilled in the art as outlined in the appended claims.

### REFERENCES

[1] Futier E, Lefrant J Y, Guinot P G, Godet T, Lorne E, Cuvillon P, Bertran S, Leone M, Pastene B, Piriou V, Molliex S, Albanese J, Julia J M, Tavernier B, Imhoff E, Bazin J E, Constantin J M, Pereira B, Jaber S and Group IS. Effect of Individualized vs Standard Blood Pressure Management Strategies on Postoperative Organ Dysfunction Among High-Risk Patients Undergoing Major Surgery: A Randomized Clinical Trial. JAMA. 2017; 318:1346-1357.
[2] Sun LY. Preoperative Risk, Blood Pressure, and Acute Kidney Injury. Anesthesiology. 2020; 132:416-417.
[3] Sun L Y, Chung A M, Farkouh M E, van Diepen S, Weinberger J, Bourke M and Ruel M. Defining an Intraoperative Hypotension Threshold in Association with Stroke in Cardiac Surgery. Anesthesiology. 2018; 129:440-447.
[4] Ngu J M C, Jabagi H, Chung A M, Boodhwani M, Ruel M, Bourke M and Sun L Y. Defining an Intraoperative Hypotension Threshold in Association with De Novo Renal Replacement Therapy after Cardiac Surgery. Anesthesiology. 2020; 132:1447-1457.
[5] Ristovic V, de Roock S, Mesana T G, van Diepen S and Sun L Y. The Impact of Preoperative Risk on the Association between Hypotension and Mortality after Cardiac Surgery: An Observational Study. J Clin Med. 2020; 9.
[6] Goldberger A L, Amaral L A, Glass L, Hausdorff J M, Ivanov P C, Mark R G, Mietus J E, Moody G B, Peng C K and Stanley H E. PhysioBank, PhysioToolkit, and PhysioNet: components of a new research resource for complex physiologic signals. Circulation. 2000; 101:E215-20.

1. A system for predicting hemodynamic events, the system comprising:
a processor;
a communications module coupled to the processor; and
a memory, the memory storing one or more trained models and computer executable instructions for predicting hemodynamic events and generating alerts to be displayed in a user interface, the computer executable instructions comprising instructions that when executed by the processor cause the system to:
receive via the communications module, current patient data;
obtain a current mean arterial pressure (MAP) value from the current patient data;
obtain prior MAP data obtained from the patient during a period of care;
use the current MAP value, the past MAP data, a predetermined MAP threshold, and at least one trained model to predict a hemodynamic event, each trained model corresponding to a prediction interval to determine whether the hemodynamic event is expected to occur within a window of time; and
output an alert indicative of the hemodynamic event to a device configured to display a graphical user interface comprising the alert.

2. The system of claim 1, wherein the at least one trained model used to predict the hemodynamic event is generated as a deep learning long-short term memory (LSTM) model.

3. The system of claim 2, wherein the LSTM model is generated using longitudinal sequential patient level data collected over time across a monitored period.

4. The system of claim 3, wherein the LSTM utilizes a sequence of hemodynamic data points in an observation window.

5. The system of claim 1, wherein the alert is displayed in the graphical user interface with a MAP trace charted over time showing a current reading and a predicted future trace relative to the predetermine MAP threshold.

6. The system of claim 1, wherein the system is remotely coupled to a monitored site via a communication network and the communication module, and the patient data is received via the communication network.

7. The system of claim 6, wherein the alert is sent via the communication network to the monitored site to be displayed by a device on or near a monitored patient.

8. The system of claim 6, wherein the alert is sent via the communication network to a client device used by a caregiver or clinician, the client device being mobile relative to the monitored site.

9. The system of claim 6, wherein the communication network is a local area network at a clinical site or a wide area network connectable to one or more local area networks.

10. The system of claim 1, wherein the system is embedded in a client device on or near a monitored patient.

11. The system of claim 1, wherein the current patient data is obtained from a patient in an intensive care unit (ICU), an operating room (OR), a critical response unit, or an inpatient ward.

12. A method of predicting hemodynamic events, the method comprising:
receiving current patient data;
obtaining a current mean arterial pressure (MAP) value from the current patient data;
obtaining prior MAP data obtained from the patient during a period of care;
using the current MAP value, the past MAP data, a predetermined MAP threshold, and at least one trained model to predict a hemodynamic event, each trained model corresponding to a prediction interval to determine whether the hemodynamic event is expected to occur within a window of time; and
outputting an alert indicative of the hemodynamic event to a device configured to display a graphical user interface comprising the alert.

13. The method of claim 12, wherein the at least one trained model used to predict the hemodynamic event is generated as a deep learning long-short term memory (LSTM) model.

14. The method of claim 13, wherein the LSTM model is generated using longitudinal sequential patient level data collected over time across a monitored period.

15. The method of claim 14, wherein the LSTM utilizes a sequence of hemodynamic data points in an observation window.

16. The method of claim 12, wherein the alert is displayed in the graphical user interface with a MAP trace charted over time showing a current reading and a predicted future trace relative to the predetermine MAP threshold.

17. The method of claim 12, wherein the system is remotely coupled to a monitored site via a communication network, and the patient data is received via the communication network.

18. The method of claim 17, wherein the alert is sent via the communication network to the monitored site to be displayed by a device on or near a monitored patient.

19. The method of claim 17, wherein the alert is sent via the communication network to a client device used by a caregiver or clinician, the client device being mobile relative to the monitored site.

20. The method of claim 17, wherein the communication network is a local area network at a clinical site or a wide area network connectable to one or more local area networks.

21. The method of claim 12, wherein the system is embedded in a client device on or near a monitored patient.

22. The method of claim 12, wherein the current patient data is obtained from a patient in an intensive care unit (ICU), an operating room (OR), a critical response unit, or an inpatient ward.

23. A non-transitory computer readable storage medium storing computer executable instructions for predicting hemodynamic events, the computer executable instructions comprising instructions for:
receiving current patient data;
obtaining a current mean arterial pressure (MAP) value from the current patient data;
obtaining prior MAP data obtained from the patient during a period of care;
using the current MAP value, the past MAP data, a predetermined MAP threshold, and at least one trained model to predict a hemodynamic event, each trained model corresponding to a prediction interval to determine whether the hemodynamic event is expected to occur within a window of time; and
outputting an alert indicative of the hemodynamic event to a device configured to display a graphical user interface comprising the alert.
